(19) United States
(12) Patent Application Publication
(10) Pub. No.: US 2025/0325792 A1
(43) Pub. Date: Oct. 23, 2025

(54) AGENT DELIVERY DEVICE

(71) Applicant: Boston Scientific Scimed, Inc., Maple Grove, MN (US)

(72) Inventors: Daniel CONGDON, Hudson, MA (US); Matthew Robert JAGELSKI, Marlborough, MA (US); Laurie A. LEHTINEN, Boylston, MA (US); Andrew PIC, Northboro, MA (US)

(73) Assignee: Boston Scientific Scimed, Inc., Maple Grove, MN (US)

(21) Appl. No.: 19/252,032

(22) Filed: Jun. 27, 2025

Related U.S. Application Data
(63) Continuation of application No. 17/123,708, filed on Dec. 16, 2020, now Pat. No. 12,370,353.
(60) Provisional application No. 62/951,426, filed on Dec. 20, 2019.

(51) Int. Cl. A61M 37/00; A61M 5/155; A61M 5/315; A61M 39/22

(52) U.S. Cl. CPC A61M 37/0069; A61M 5/155; A61M 5/31586; A61M 39/22; A61M 2205/8206

(57) ABSTRACT

A medical device comprising a housing defining at least one enclosure for storing agent in a first form, a force applicator within the housing and adjacent the enclosure, a drive mechanism for moving the agent toward the force applicator, wherein the force applicator defines a surface for applying a force to the agent to separate the agent into particles smaller than a size of the first form, and wherein the device defines a lumen for receiving the particles from the force applicator and for receiving a pressurized fluid to propel the particles through the lumen.

### CROSS-REFERENCE TO RELATED APPLICATIONS

The present application is a continuation of U.S. application Ser. No. 17/123,708, filed on Dec. 16, 2020, which claims the benefit of priority under 35 U.S.C. § 119 to U.S. Provisional Application No. 62/951,426, filed on Dec. 20, 2019, each of which is incorporated by reference in its entirety.

### TECHNICAL FIELD

The present disclosure relates generally to a medical device that administers an agent. More particularly, at least some embodiments of the present disclosure relate to a medical device configured to be loaded with a therapeutic agent, separate the loaded agent into a smaller form, and then deliver that agent via a lumen of the medical device.

### BACKGROUND

In certain medical procedures, it may be necessary to stop bleeding internal to the body. For example, an endoscopic medical procedure may require hemostasis of bleeding tissue within the gastrointestinal tract, for example in the esophagus, stomach, or intestines.

During an endoscopic procedure, a user inserts a sheath of an endoscope into a body lumen of a patient. The user utilizes a handle of the endoscope to control the endoscope during the procedure. Tools are passed through a working channel of the endoscope via, for example, a port in the handle, to deliver treatment at the procedure site near a distal end of the endoscope. The procedure site is remote from the operator.

To achieve hemostasis at the remote site, a hemostatic agent may be delivered by a device inserted into the working channel of the endoscope. Agent delivery may be achieved through mechanical systems, for example. Such systems, however, may require numerous steps or actuations to achieve delivery, may not achieve a desired rate of agent delivery or a desired dosage of agent, may result in the agent clogging portions of the delivery device, may result in inconsistent dosing of agent, or may not result in the agent reaching the treatment site deep within the GI tract. The current disclosure may solve one or more of these issues or other issues in the art.

### SUMMARY OF THE DISCLOSURE

According to an example, a medical device may include a housing defining at least one enclosure for storing agent in a first form, a force applicator within the housing and adjacent the enclosure, a drive mechanism for moving the agent toward the force applicator. The force applicator may define a surface for applying a force to the agent to separate the agent into particles smaller than a size of the first form. The device may define a lumen for receiving the particles from the force applicator and for receiving a pressurized fluid to propel the particles through the lumen. The force applicator may include a round gear including a plurality of teeth about a circumference of the gear. The drive mechanism may include two rotatable wheels to receive the agent between the two rotatable wheels, and may be connected to a trigger outside the housing. The actuation of the trigger may cause rotation of the two rotatable wheels.

In another example, the medical device may further include a fluid source, e.g., gas, for providing the pressurized fluid, wherein the fluid source may be connected to the lumen via a fluid channel, and wherein the fluid channel may be connected to a portion of the lumen distal to the force applicator. The fluid channel may include a valve configured to open or close a flow of pressurized fluid from the fluid source to the lumen. The valve may coupled to the trigger configured to at least open/close the valve.

In another example, the medical device may further include a first gear coupled to a surface of the force applicator, wherein the first gear is configured to rotate simultaneously with the force applicator, in a same direction, and a lever coupled to the housing, wherein an end of the lever includes a second gear, and the second gear and the first gear are connected in series via a linking gear positioned in between the first gear and the second gear, wherein actuation of the lever rotates the second gear, which rotates the linking gear, which rotates the first gear and the force applicator. Actuation of the lever may also actuate a trigger to supply a pressurized fluid to the lumen. Each throw, e.g., pivoted rotation, of the lever may rotate the force applicator by a consistent degree to supply a substantially consistent amount of particles to the lumen.

In another example, the medical device may further include an electric motor coupled to the force applicator, wherein the electric motor is configured to rotate the force applicator, and a battery electrically connected to the electric motor and a trigger, wherein the trigger is configured to act as an electrical switch that powers the electric motor via the battery. Actuation of the trigger may continuously rotate the force applicator and continuously supply pressurized fluid to the lumen from the fluid source, until the trigger is released.

In another example, the housing of the medical device may include a holster defining a plurality of enclosures for storing the agent, wherein the holster is rotatable relative to other portions of the housing and the lumen. The housing may include a chamber below the holster, and a channel between the chamber and the lumen so that there is fluid communication between the chamber and the lumen. The drive mechanism may include a rotation of the holster so that one of the plurality of enclosures aligns with the chamber, thereby delivering the agent from one of the enclosures to the chamber. The force applicator may include a wedge that obtrudes into the chamber, and the wedge may be configured to separate the agent in the chamber into particles.

According to another example, a medical device may include a housing defining at least one enclosure for storing agent in a first form, a force applicator within the housing and adjacent the enclosure, a drive mechanism for moving the agent toward the force applicator, wherein the force applicator includes a plurality of teeth for applying a force to the agent to separate the agent into particles smaller than a size of the first form, wherein the force applicator includes a first gear, and a lever coupled to the housing. The lever may include a second gear, and the second gear and the first gear may be coupled so that pulling the lever causes the second gear to rotate the first gear and the force applicator, separating the agent into the particles. The device may define a lumen for receiving the particles from the force applicator and for receiving a pressurized fluid to propel the particles through the lumen. The drive mechanism may be connected to a trigger outside the housing, and actuation of the trigger may operate the drive mechanism.

In another example, the medical device may further include a fluid source for providing the pressurized fluid, wherein the fluid source is connected to the lumen via a fluid channel, wherein the fluid channel includes a valve configured to open or close a flow of pressurized fluid from the fluid source to the lumen, wherein the valve is coupled to a trigger configured to at least open/close the valve, and wherein the trigger is located outside of the housing. Actuation of the lever may actuate a trigger to supply the pressurized fluid to the lumen.

According to an example, a method of administering agent via a medical device may include positioning a lumen of the medical device so that a distal end of the lumen is adjacent to a targeted site, wherein the device further includes a housing defining at least one enclosure storing the agent in a first form, a force applicator within the housing and adjacent the enclosure, and a drive mechanism for moving the agent toward the force applicator, providing a pressurized fluid to the lumen, and delivering the agent towards the force applicator via the drive mechanism, thereby separating the agent into particles smaller than a size of the first form via the force applicator, and feeding the lumen with the particles.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A-1B are cross-sectional views of a medical device, according to different embodiments.

FIG. 2 is a cross-sectional view of an enclosure and a feeding mechanism of a medical device, according to an embodiment.

FIG. 3 is a cross-sectional view of a portion of a medical device, according to another embodiment.

FIG. 4A is a cross-sectional view of a medical device, according to another embodiment.

FIG. 4B is a top view of a holster of the medical device of FIG. 4A.

FIGS. 4C and 4D are cross-sectional views of a medical device, according to another embodiment.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic.

The present disclosure may solve one or more of the limitations in the art. The scope of the disclosure, however, is defined by the attached claims and not the ability to solve a specific problem. The present disclosure is drawn to medical devices configured to be loaded with agent(s), e.g., therapeutic agents, that crush or separate the loaded agent(s) into smaller particles, and administer said particles to a targeted site, among other aspects. The agent may be in any first form, e.g., a rod, a pellet, prior to it being separated or crushed into a smaller form, such as a powder of loose particles, and delivered to a lumen receiving a stream of propellant/pressurized fluid, e.g., CO₂, nitrogen, air, etc. Said medical devices may help increase the consistency of particle size and particle delivery of agent, e.g., hemostatic powder, and may also help reduce variation that is inherent in conventional fluid-driven powder/particle mixing and delivery systems.

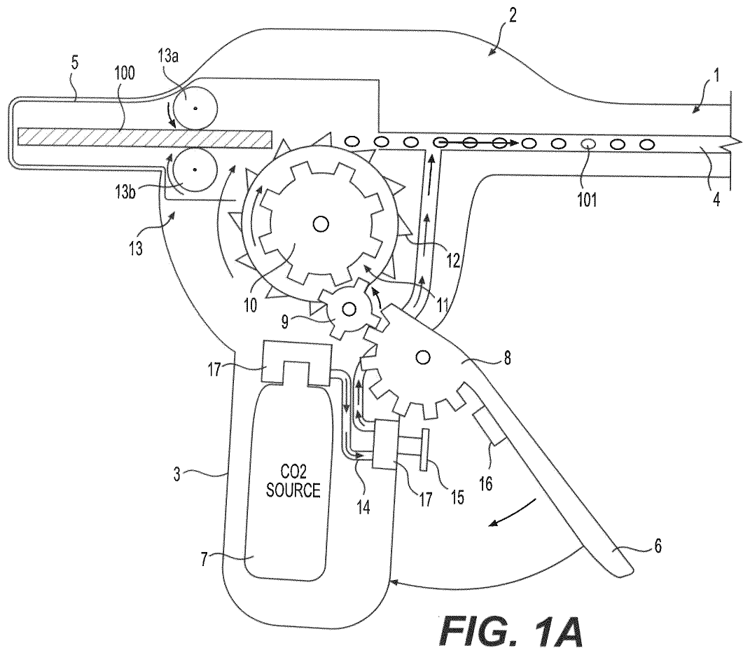

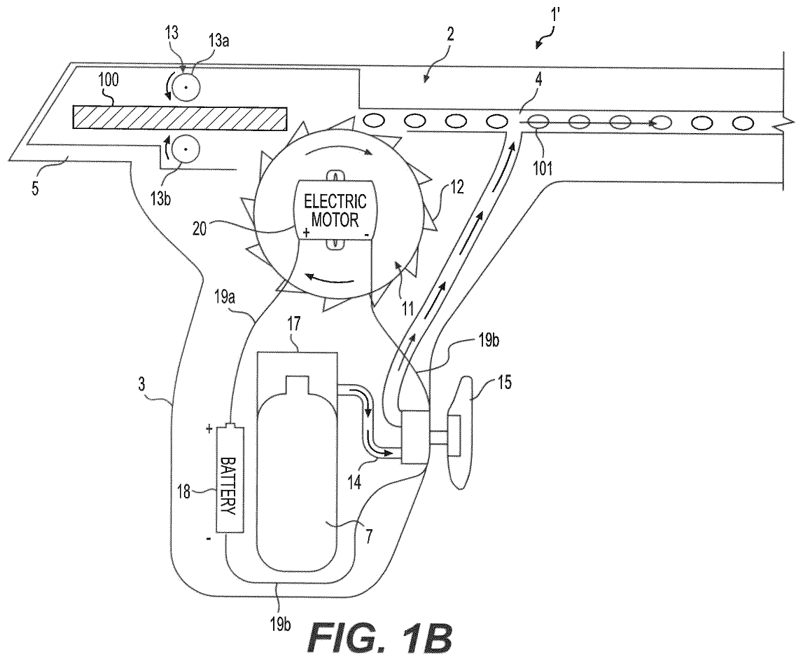

FIG. 1A illustrates an exemplary embodiment of medical device 1 in further detail. Medical device 1 includes a housing 2 defining at least one enclosure 5 for storing agent 100 in a first form, e.g., a rod or other single-piece shaped form of an agent, a force applicator 11 within housing 2 and adjacent enclosure 5, and a drive mechanism 13 configured for moving or propelling agent 100 towards force applicator 11. Enclosure 5 may be pre-loaded with agent 100, or enclosure 5 may include an opening/mechanism by which it may be loaded with agent 100. Force applicator 11 defines a surface, e.g., a surface of each of a plurality of teeth 12 along its circumference, for applying a force to agent 100 to separate it into particles 101 smaller than its first form. Instead of teeth 12 about its circumference, force applicator 11 could include serrations, barbs, or any other sharp or roughened surface capable of separating agent into powder/particle form. Drive mechanism 13 includes two wheels 13a and 13b positioned directly below and above one another, with sufficient space between one another to receive agent 100. Drive mechanism 13 may be within enclosure 5, in which agent 100 is stored. Drive mechanism 13 moves agent 100 towards force applicator 11 as wheel 13a rotates counter-clockwise and wheel 13b rotates clockwise. However, drive mechanism 13 is not limited to wheels 13a and 13b, and may be any suitable mechanism for advancing agent 100. Similarly, force applicator 11 may be any suitable mechanism for separating/crushing agent 100 into particles 101, such as, round grinders, worm gears, or augers. In some other embodiments, device 1 may include a plurality of force applicators.

Medical device 1 also includes a lumen 4 within housing 2 for receiving particles 101 from force applicator 11 and for receiving a pressurized fluid from a fluid source 7, via a channel 14, to propel particles 101 through lumen 4. Lumen 4 may be connected with or otherwise be in fluid communication with enclosure 5 storing agent 100, so that a distal portion of enclosure 5 transitions into lumen 4, as shown in FIG. 1A. Channel 14 includes a valve 17 along its length between source 7 and lumen 4, and is connected to a portion of lumen 4 that is distal to force applicator 11 to feed lumen 4 with pressurized fluid, thereby propelling particles 101 towards a distal end of lumen 4. Valve 14 is coupled to a trigger 15 located outside of a handle portion 3 of housing 2. Trigger 15 may be configured to at least open fluid valve 14, thereby providing a flow of pressurized fluid from fluid source 7 to lumen 4. Trigger 15 may be any suitable form, e.g., a button, a switch, and its form is not particularly limited. Fluid source 7 is within handle portion 3 of housing 2, but is not particularly limited to being within handle 3, and may even be outside housing 2. It is further noted that lumen 4 is not limited to being within housing 2, and in other embodiments, may be at least partially outside of housing 2. A catheter/sheath (not shown) may also be attached to (or otherwise extend from) the distal end of housing 2. Said catheter/sheath may be long, flexible to traverse tortuous patient anatomy, and any suitable size to insert into a working lumen of a scope (not shown) or another delivery device (not shown).

Medical device 1, shown in FIG. 1A, further includes a first gear 10 that is coupled to a surface of force applicator 11, and first gear 10 is configured to rotate simultaneously with force applicator 11, in the same direction. Medical device 1 also includes a lever 6 coupled to housing 2, and the coupled end of lever 6 includes a second gear 8. First gear 10 and second gear 8 are connected in series via a linking gear 9, positioned in between first gear 10 and second gear 8. Thus, lever 6 is pivotably coupled to linking gear 9, via second gear 8. As a result of such configuration, pulling lever 6 proximally rotates second gear 8 (clockwise as shown in FIG. 1A), which in turn rotates linking gear 9 (counter-clockwise), which rotates first gear 10 (clockwise) by a selected or desired degree. Pulling lever 6 proximally may entail translating one end of lever 6 (the end opposite of second gear 8) towards handle portion 3, as indicated by the directional arrow of FIG. 1A. This translation is a pivoted movement, as the other end of lever 6 (second gear 8) is pivotably connected to linking gear 9. The pulling of lever 6 may be by any suitable action, for example, by hand or by mechanical, electrical, or pneumatic action. The rotation of first gear 10 rotates force applicator 11 by a selected or desired degree, which proceeds to separate/crush a portion of agent 100, fed via drive wheels 13a and 13b, into particles 101. It is noted that the rotation of drive wheels 13a and 13b may be actuated via any suitable mechanism. Such mechanisms may include an automated mechanism that is triggered by the pulling of lever 6 or by the actuation of trigger 15 opening valve 17. Another mechanism may include an additional gear or a series of gears connecting first gear 10 to drive wheels 13a and 13b, and thus, also rotating said drive wheels by pulling lever 6.

Lever 6 further includes a contact 16, which may be a protrusion or a tab extending outward from the surface of lever 6 facing handle 3. Contact 16 may be configured to press or actuate trigger 15 on handle 3, as lever 6 is pulled proximally towards handle 3, thereby opening valve 17. Opening valve 17 permits pressurized fluid to flow into channel 14 past valve 17 and into lumen 4 to propel particles 101 distally. Referring to FIG. 1A, an example of how medical device 1 may be used is further discussed below. The distal portion of medical device 1 (e.g., a catheter or sheath having the distal portion of lumen 4) may be delivered into the body of a subject. Lumen 4 may be positioned so that a distal end of lumen 4 is adjacent to an intended target site for agent 100 administration. Such delivery and positioning may be accomplished via an endoscope having a working channel (not shown). Imaging associated with the endoscope may assist in positioning. A user may then load housing 2 of medical device 1 with agent 100, if not loaded already, so that wheels 13a and 13b of driving mechanism 13 may advance agent 100 towards force applicator 11. The manner in which housing 2 is loaded with agent 100 is not particularly limited. A user may then pull lever 6 proximally, by any suitable manner/mechanism, so that contact 16 presses against trigger 15, thereby opening valve 14 and providing a pressurized fluid to lumen 4, and also rotating force applicator 11 via the series of first gear 10, linking gear 9, and second gear 8. This results in force applicator 11 separating/crushing agent 100 into particles 101, via the force applied by teeth 12 to agent 100. Lumen 4 is fed with particles 101, which are propelled toward the distal end of lumen 4 via pressurized fluid and, thus, administered to the intended target site. Pulling lever 6 may also trigger a mechanism by which driving wheels 13a and 13b are rotated, such as via automated rotation or via a gear or a series of additional gears connecting first gear 10 to driving wheels 13a and 13b.

It is further noted that a throw of lever 6 rotates force applicator 11 by a certain degree, as discussed above, and supplies a continuous flow of pressurized fluid, as long as trigger 15 is pressed inward. Thus, a single throw of lever 6 may deliver a consistent, desired dose of agent 100, separated into particles 101, into lumen 4. For example, FIG. 1A shows that lever 6 may be rotated about 45 degrees clockwise, causing a set degree of rotation of gears 9, 10 and force applicator 11. This set amount of rotation corresponds to a set amount of agent 100 being separated into particles. For larger doses, a user may repeatedly actuate (push then pull) lever 6 so that force applicator 11 is intermittently rotated and pressurized fluid is supplied. Alternatively, for larger doses, the throw of lever 6 may be increased.

Medical device 1′, as shown in FIG. 1B, is similar to device 1 in many respects. Like reference numeral refer to like parts. Differences between device 1 and 1′ will be described. Device 1′ includes an electric motor 20 coupled to force applicator 11, configured to rotate force applicator 11 electrically. In some other embodiments, medical device l′ may include a plurality of electric motors 20 and/or a plurality of force applicators 11. Medical device l′ further includes a battery 18 electrically connected to electric motor 20, via wires 19a and 19b, to power electric motor 20. Wire 19a connects the cathode of battery 18 to electric motor 20. Wire 19b includes two portions to connect the anode of battery 18 and motor 20 to trigger 15′, which serves as both a valve switch and an electrical switch, along its path to electric motor 18. Therefore, the actuation of trigger 15′ may open valve 17 and power electric motor 20 simultaneously in medical device 1′. The actuation of trigger 15′ may be by any suitable action, for example, by hand or by mechanical, electrical, or pneumatic action.

Medical device l′ may be used in the same manner as medical device 1 except a user actuates trigger 15′, as opposed to pulling a lever. The actuation of trigger 15′ opens valve 14, thereby providing a pressurized fluid to lumen 4, and also powers the rotation of force applicator 11 via electric motor 20, which is electrically wired to battery 18. Additionally, the actuation of trigger 15′ may also automate the rotation of driving wheels 13a and 13b, thereby feeding agent 100 to force applicator 11. Actuation of trigger 15′ may operate the aforementioned functions in a continuous manner, until trigger 15′ is released. Thus, a user may hold trigger 15′ until a desired amount of agent 100, separated into particles 101, is delivered to a targeted site via lumen 4, and then release trigger 15′ to cease the operation of device l′.

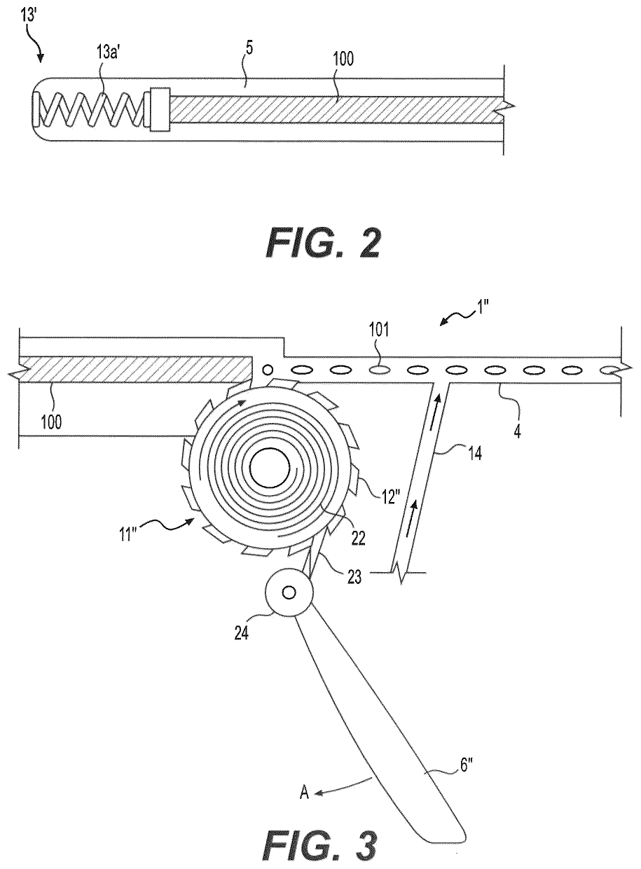

FIG. 2 shows an example of another embodiment of drive mechanism 13′. Drive mechanism 13′ includes a compressed spring 13a′ coupled to a platform 13b′ on one end, that is positioned adjacent to a proximal end of agent 100. Platform 13b′ defines a surface which pushes against agent 100, as compressed spring 13a′ decompresses, thereby pushing agent 100 towards a force applicator (not shown). The other end of spring 13a′ is stationary against an inner surface of enclosure 5 in the housing. Agent 100 may be pre-loaded into enclosure 5, or enclosure 5 may include an opening/mechanism by which it may be loaded with agent 100. In this embodiment, a surface of one of the teeth, or any other force applying surface, of a force applicator (not shown) may apply an opposite, greater force against spring 13a′, so that spring 13a′ remains compressed and agent 100 is not advanced. By such configuration, spring 13a′ extends and agent 100 is advanced only when the force applicator is rotated or actuated.

FIG. 3 shows an example of another means by which force applicator 11 may be rotated in medical device 1″ to apply a force separating/crushing agent 100 into particles 101. In medical device 1″, a torsion spring 22 is coupled to force applicator 11 in a manner so that a torque or a rotary force actuates the rotation of force applicator 11″. Medical device 1″ further includes a lever 6″ configured to pivot about a pivot point 24. Lever 6″ includes a pawl 23 that may catch one of teeth 12″ of force applicator 11″, thereby inhibiting the rotation of force applicator 11″. As lever 6″ is pulled proximally (as shown by the arrow A), and pivots clockwise about pivot point 24, pawl 23 simultaneously rotates in a clockwise direction, and releases from one of teeth 12″ of force applicator 11″, thereby rotating force applicator 11″ via the rotary force exerted by spring 22. As shown, the rotation of force applicator 11″ applies a force to agent 100 via teeth 12″, and separates/crushes agent 100 into particles 101, which are subsequently delivered to lumen 4. Thus, medical device 1″ may be used in a similar manner as medical device 1. Medical device 1″ may also be different in use than device 1. For example, lever 6″ may be pulled (as shown by arrow A) and held in its pulled position to permit continuous rotation of force applicator 11′, via the rotary force exerted by spring 22, and to have a constant supply of pressurized fluid. Thus, unlike device 1, multiple, sequential throws of lever 6″ is not necessary to continually rotate force applicator 1″ and supply pressurized fluid for a prolonged duration of time. Lever 6″ may also be returned to its original position so that pawl 23 re-engages one of teeth 12″ to inhibit further rotation of force applicator 11″, and to also cease the supply of pressurized fluid to lumen 4. Lever 6″ may be actuated by any suitable action, for example, by hand or by mechanical, electrical, or pneumatic action.

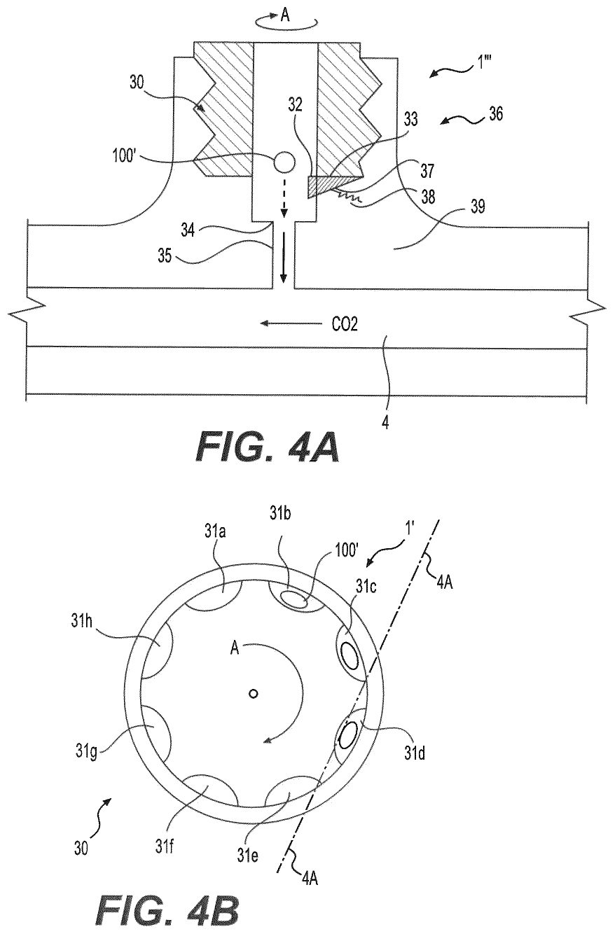

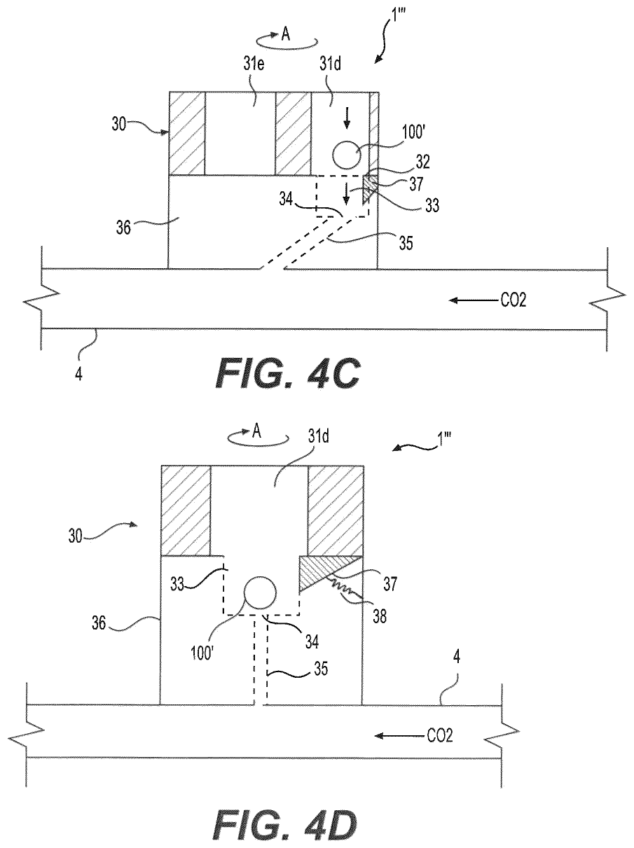

Referring to FIGS. 4A-4D, another embodiment of medical device 1′″ is described below. Medical device 1′″ includes a housing 36 that includes a holster 30, which includes a plurality of cavities 31a-h for storing agent 100′ in a first form, e.g., a pellet. Holster 30 sits within a enclosure of housing 36. FIG. 4A shows a cross-sectional view of holster 30 along line 4A-4A of FIG. 4B. Medical device 1′″ also includes a lumen 4 receiving pressurized fluid, e.g., CO₂, from a fluid source (not shown) at its proximal end. Housing 36 includes a barrier region 39 positioned between holster 30 and lumen 4. As indicated by the directional arrow A in FIGS. 4A and 4B, holster 30 is rotatable relative to a remainder of housing 36 and lumen 4. Furthermore, housing 36 includes a force applicator in the form of a wedge 37, which defines a surface for applying a force to agent 100 to separate it into particles smaller than its first form. The form of a force applicator is not particularly limited to wedge 37, and may be any suitable form. Wedge 37 may be below holster 30, and may be spring-actuated, via spring 38. Any other form of biasing or pressing wedge 37 to the left in FIG. 1A may be used. In another embodiment, wedge 37 may be actuated via pneumatics. For example, an additional port or channel (not shown) may be branched from lumen 4 at a point that is proximal to a channel 35, so that said port may feed pressurized fluid directly into housing 36 or specifically towards wedge 37. The force of the fed pressurized fluid may engage wedge 37 to compress and crush agent 100′. In other embodiments, a combination of both a spring and pneumatics may be implemented to actuate wedge 37.

Housing 36 also includes a chamber 33 defined by a first opening 32 that is adjacent to holster 30, and a narrower, second opening 34 leading to a channel 35, which leads to lumen 4. First opening 32 may be aligned with any of the plurality of enclosures 31a-h of holster 30, depending on the rotational position of holster 30 relative to barrier 39. Thus, as holster 30 rotates, agent 100′ may drop into chamber 33 from one of the plurality of enclosures 31a-h. The rotation of holster 30 may be by any suitable action, for example, by hand or by mechanical, electrical, or pneumatic action. For example, in some other embodiments, the rotation of holster 30 may be operated by a trigger that causes rotation or measured rotation of holster 30, e.g., rotation such that adjacent enclosures 31a-31h may be aligned with chamber 33 sequentially. In FIGS. 4A-4B, holster 30 includes eight enclosures 31a-h distributed evenly about the perimeter/circumference of holster 30. Actuation of a trigger may cause rotation of holster by 45 degrees to align a subsequent enclosure with opening 32. Although eight equally sized and spaced enclosures are shown, it is understood that there may be more or less number of enclosures, varied spacing, and varied size to accommodate different sizes/doses of agent 100′.

A portion of wedge 37, which may be spring-actuated via spring 38, obtrudes the enclosure defined by chamber 33, and after holster 30 releases agent 100′ into chamber 33, wedge 37 separates/crushes agent 100′ into particles (not shown). Because there is fluid communication between chamber 33 and lumen 4, via channel 35, the particles of agent 100′ are delivered to lumen 4, and are propelled towards a distal end of lumen 4 via pressurized fluid. It is noted that agent 100′, prior to being separated into particles, is inhibited from falling into channel 35 and being delivered to lumen 4 because second opening 34 and channel 35 are narrower than a width, or cross-sectional size, of agent 100′, in whichever first form. A size of opening 34 and channel 35, and a force exerted by wedge 37, may control the size of particles delivered.

Referring to FIGS. 4A-4D, an example of how medical device 1′″ may be used is further discussed below. Similar to the aforementioned exemplary medical devices, a distal portion of medical device 1′″ (e.g., a catheter or sheath having the distal portion of lumen 4) may be delivered into the body of a subject. Lumen 4 may be positioned/directed so that a distal end of lumen 4 is adjacent an intended target site for agent 100 administration. As previously discussed, such delivery and positioning may be accomplished via an endoscope having a working channel (not shown). Imaging associated with the endoscope may assist in positioning. A user may then load one or more of the plurality of enclosures 31a-h of holster 30 with agent 100′, if not loaded already. The user may then rotate holster 30 relative a remainder of housing 36 and lumen 4 so that one of enclosures 31a-31h aligns with first opening 32, thereby dropping agent 100′ into chamber 33. Wedge 37 proceeds to apply a force onto agent 100′, thereby separating/crushing agent 100′ into particles (not shown). Rotation of holster 30 may be by any suitable manner or mechanism, e.g., by hand or by mechanical, electrical, or pneumatic action. Said rotation may also be a measured rotation or a continuous rotation via a mechanical or electrical means. Because there is fluid communication between chamber 33 and lumen 4, via channel 35, said particles are delivered to lumen 4, and are propelled towards a distal end of lumen 4 via pressurized fluid. It is noted that pressurized fluid may be supplied to lumen 4, by a fluid source, at any time prior to, during, and after the rotation of holster 30.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
a housing including an enclosure for storing an agent;
an inlet configured to receive a pressurized fluid, the inlet in fluid communication with an outlet;
a movable body at least partially disposed within the enclosure; and
an actuation mechanism configured to rotate the movable body within the enclosure as the inlet guides the pressurized fluid by the movable device and towards the outlet, such that the movable body is configured to move the agent from the enclosure towards the outlet to mix with the pressurized fluid.

2. The medical device of claim 1, wherein the movable body includes at least one protrusion extending radially outwards from an exterior surface of the movable body.

3. The medical device of claim 1, wherein the movable body is configured to move between a first configuration and a second configuration upon actuation of the actuation mechanism; and
wherein in the first configuration, the enclosure is not in fluid communication with the outlet; and
wherein in the second configuration, the enclosure is in fluid communication with the outlet.

4. The medical device of claim 2, wherein the at least one protrusion is disposed within the enclosure and configured to separate the agent from a compact state to a separated state.

5. The medical device of claim 1, wherein the enclosure includes an opening that is in fluid communication with the inlet and the outlet, and the movable body is at least partially disposed within the enclosure adjacent to the opening.

6. The medical device of claim 5, further comprising a channel disposed downstream from the enclosure and upstream from the outlet, the channel is configured to receive the agent from the opening of the enclosure upon actuation of the actuation mechanism.

7. The medical device of claim 1, wherein the actuation mechanism is configured to release the pressurized fluid into the inlet upon actuation.

8. The medical device of claim 1, wherein the actuation mechanism is configured to rotate the movable body by a predetermined degree to supply a substantially consistent volume of the agent to the outlet upon each actuation.

9. The medical device of claim 1, further comprising:
an electric motor coupled to the movable body, wherein the electric motor is configured to rotate the movable body upon activation; and
a battery electrically connected to the electric motor and a trigger, wherein the trigger is an electrical switch that is configured to power the electric motor via the battery.

10. The medical device of claim 9, wherein the trigger is configured to move the actuation mechanism, thereby causing the movable body to continuously rotate and supply a continuous flow of the pressurized fluid received via the inlet from a pressurized fluid source until released.

11. The medical device of claim 1, wherein the housing includes a chamber disposed below the enclosure and a channel disposed between the chamber and the outlet, such that the enclosure is in fluid communication with the outlet via the chamber.

12. A medical device, comprising:
at least one enclosure for storing an agent;
a chamber disposed below the at least one enclosure, the chamber configured to receive the agent from the at least one enclosure;
an inlet configured to receive pressurized fluid from a fluid source;
an outlet configured to deliver a combination of the agent and the pressurized fluid; and
a movable body disposed between the inlet and the outlet, the movable body extends at least partially into the at least one enclosure, wherein the movable body is configured to rotate relative to the at least one enclosure between a first configuration and a second configuration to release the agent stored in the at least one enclosure towards the outlet to form the combination with the pressurized fluid.

13. The medical device of claim 12, wherein the movable body includes at least one protrusion extending radially outwards from the movable body, and the at least one protrusion is disposed within the at least one enclosure and configured to separate the agent from a compact state to a separated state.

14. The medical device of claim 12, wherein when in the first configuration, the movable body is configured to rotate relative to the at least one enclosure to fluidly decouple the at least one enclosure from the outlet via the chamber.

15. The medical device of claim 12, wherein when in the second configuration, the movable body is configured to rotate relative to the at least one enclosure to fluidly couple the at least one enclosure with the outlet via the chamber, thereby releasing the agent from the at least one enclosure into the chamber for combining with the pressurized fluid to form the combination in the outlet.

16. The medical device of claim 12, further comprising an actuation mechanism configured to rotate the movable body in response to actuation of the actuation mechanism.

17. The medical device of claim 16, wherein the actuation mechanism includes a trigger, wherein actuation of the trigger simultaneously rotates the movable body and supplies the pressurized fluid into the chamber.

18. The medical device of claim 17, wherein actuation of the trigger is configured to generate a continuous flow of the pressurized fluid from the fluid source towards the chamber until released.

19. The medical device of claim 12, further comprising:
a motor coupled to the movable body, the motor is configured to rotate the movable body; and
a power source connected to the motor, the power source is configured to activate the motor upon activation, thereby rotating the movable body within the at least one enclosure.

20. A method of administering an agent via a medical device, the method comprising:
positioning a lumen of the medical device with a distal end of the lumen adjacent to a target site, wherein the medical device includes an enclosure storing the agent, a movable body extending at least partially into the enclosure, and an actuation mechanism configured to rotate the movable body;
actuating the actuation mechanism to rotate the movable body within the enclosure, thereby causing the movable body to separate the agent stored in the enclosure from a compact state to a separated state via at least one protrusion on the movable body contacting the agent;
continuing actuation of the actuation mechanism to supply a pressurized fluid for mixing with the agent in the separated state; and
delivering a mixture of the pressurized fluid and the agent in the separated state towards the lumen for delivery to the target site.
